(19) United States
(12) Patent Application Publication
(10) Pub. No.: US 2023/0141109 A1
(43) Pub. Date: May 11, 2023

(54) TOOTHPASTE CAPSULE

(71) Applicant: IGAL ARTZENU COHEN, Nesher (IL)

(72) Inventor: IGAL ARTZENU COHEN, Nesher (IL)

(21) Appl. No.: 17/981,502

(22) Filed: Nov. 7, 2022

(30) Foreign Application Priority Data
Nov. 11, 2021 (IL) 288027

(51) Int. Cl. A61K 8/11

(52) U.S. Cl. CPC A61K 8/11

(57) ABSTRACT

A toothpaste capsule, including a receptacle, a first sheet, for covering the receptacle, and being removable, for producing an opening to the receptacle, and a toothpaste dose contained between the receptacle and the first sheet.

### CROSS-REFERENCE TO RELATED APPLICATIONS

This application claims the benefit of priority from Israel Patent Application No. 288027, filed Nov. 11, 2021, the disclosure of which is incorporated herein by reference.

### TECHNICAL FIELD

The invention relates to the field of toothpaste.

### BACKGROUND

There is a long felt need to provide a convenient toothpaste capsule, as a solution to the above-mentioned and other problems of the prior art.

### SUMMARY

A toothpaste capsule, including:
a receptacle;
a sheet, for covering the receptacle; and
a toothpaste dose.

### BRIEF DESCRIPTION OF THE DRAWINGS

Embodiments, features, and aspects of the invention are described herein in conjunction with the following drawings:

FIG. 1 is a perspective view of a receptacle and a sheet.

FIG. 2 is a side view of a toothpaste capsule according to one embodiment of the invention.

FIG. 3 depicts the first step of using the toothpaste capsule of FIG. 2.

FIG. 4 depicts the second and third steps of using the toothpaste capsule of FIG. 2.

FIG. 5 depicts a chain of toothpaste capsules of FIG. 2.

FIG. 6 depicts a chain of toothpaste capsules of FIG. 2.

FIG. 7 is a side view of the chain of toothpaste capsules of FIG. 6.

FIG. 8 is a perspective view of one of the toothpaste capsules of FIG. 6.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

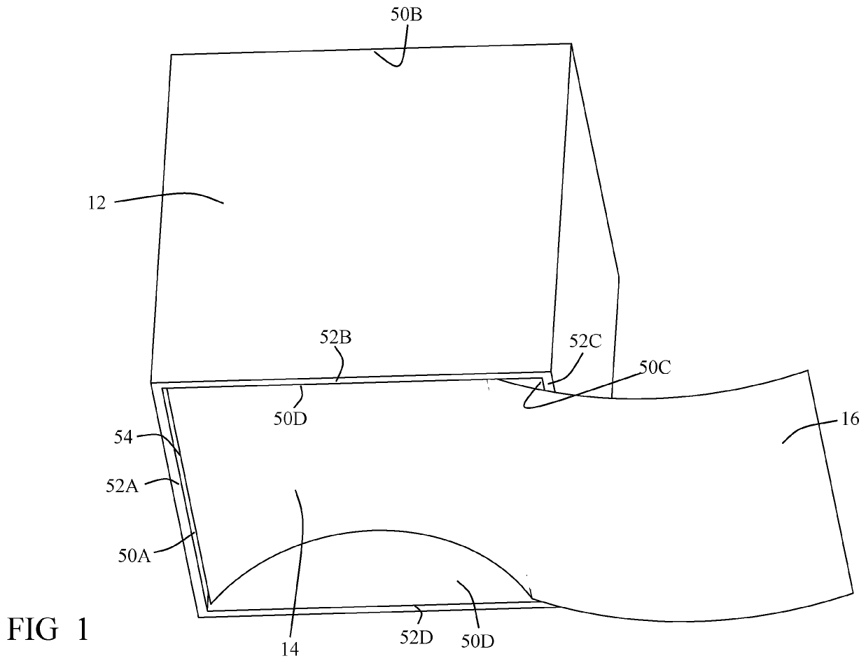

FIG. 1 is a perspective view of a receptacle and a sheet.

A toothpaste capsule 10A includes a receptacle 12; a sheet 14, a first end 54 thereof being connected to an end 52A of an internal wall 50C, thus normally attaching internal walls 50A and 50C and internal ceiling 56 of receptacle 12.

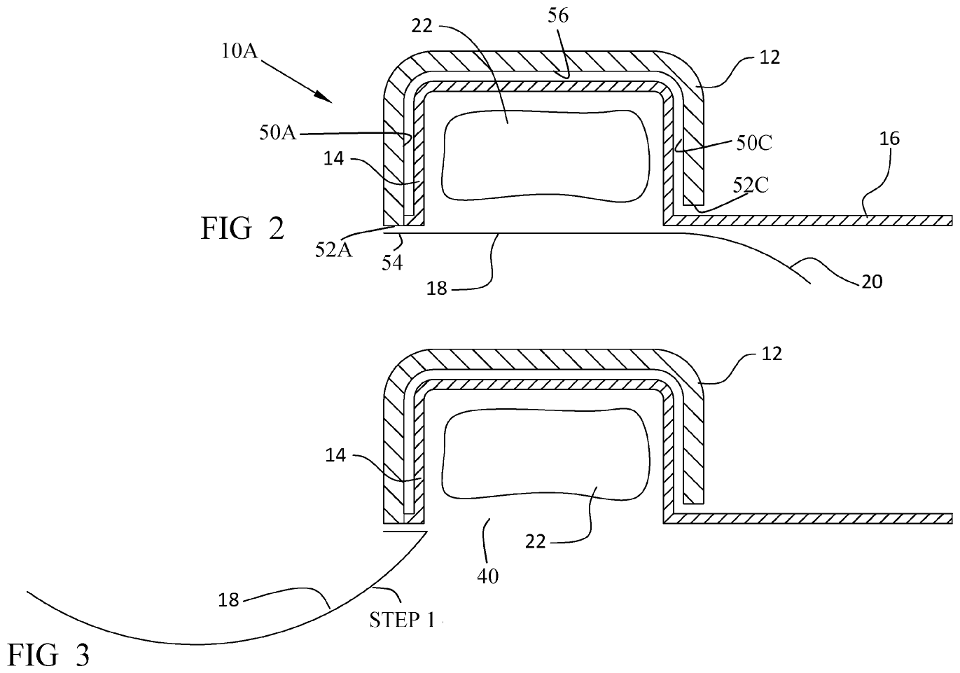

FIG. 2 is a side view of a toothpaste capsule according to one embodiment of the invention.

Sheet 14 of toothpaste capsule 10A contains a toothpaste dose 22. Toothpaste capsule 10A further includes a cover 18, for covering receptacle 12, by sticking to ends 52A,52B,52C,52D (enumerated in FIG. 1) of internal walls 50A,50B,50C,50D respectively thereof.

FIG. 3 depicts the first step of using the toothpaste capsule of FIG. 2.

At the first step, the user removes cover 18, thus canceling holding of sheet 14 to receptacle 12; and further producing an opening 40 to toothpaste dose 12.

Cover 14 may be a foil, such as of aluminum.

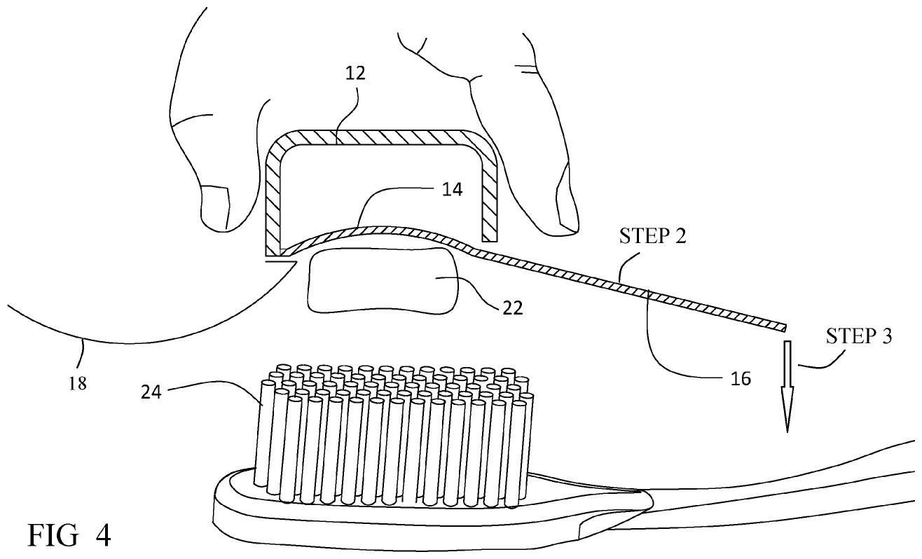

FIG. 4 depicts the second and third steps of using the toothpaste capsule of FIG. 2.

At the second step, sheet straightens, upon being freed from the cover 18.

At the third step, the straightening of sheet 14 which may be accompanied by moving the tail 16 of sheet 14, pushes toothpaste dose 12 onto a toothbrush 24, without squashing toothpaste dose 12 or changing the original shape thereof.

The pushing operation of toothpaste dose 12 allows removal thereof from receptacle 14, even if being rigid, without squeezing toothpaste dose 12.

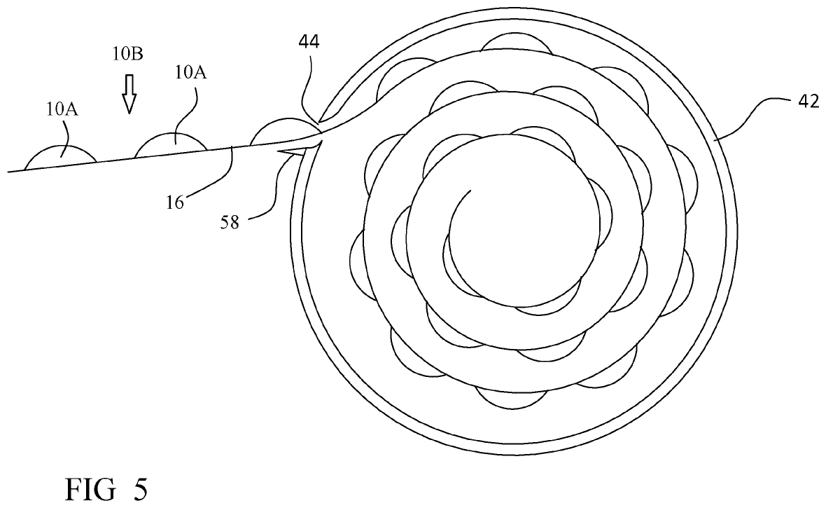

FIG. 5 depicts a chain of toothpaste capsules of FIG. 2.

A toothpaste capsule chain 10B may include a plurality of toothpaste capsules 10A being chained one to the other.

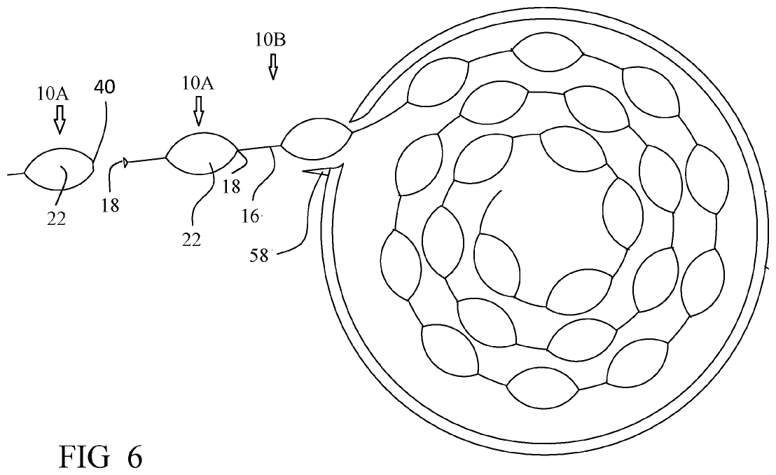

FIG. 6 depicts a chain of toothpaste capsules of FIG. 2.

Cover 18 of each toothpaste capsule 10A may be disposed between each adjacent toothpaste capsules 10A, thereby separation of toothpaste capsule 10A from the adjacent toothpaste capsule 10A produces opening 40.

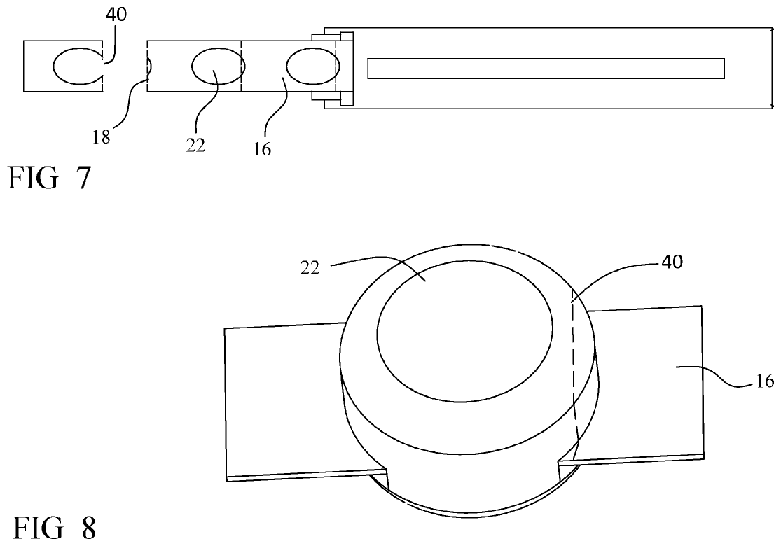

FIG. 7 is a side view of the chain of toothpaste capsules of FIG. 6.

Thus, the chaining produces opening 40 of each toothpaste capsule 10A upon removing each toothpaste capsule 10A from toothpaste capsule chain 10B.

FIG. 8 is a perspective view of one of the toothpaste capsules of FIG. 6.

Thus, in one aspect, the invention is directed to a toothpaste capsule (10A), including:
a receptacle (12);
a first sheet (18), for covering the receptacle (12), and being removable, for producing an opening (40) to the receptacle (12); and
a toothpaste dose (22) contained between the receptacle (12) and the first sheet (18).

The toothpaste capsule (10A) may further include:
a second sheet (14), a first end (54) thereof being connected to an end (52) of one (50A) of internal walls (50A,50C) of the receptacle (12), the second sheet (14) for surrounding the toothpaste dose (22),
thereby removal of the first sheet (18) allows straightening the second (14), to push the toothpaste dose (22) out of the receptacle (12).

The second sheet (14) may include a tail (16), for accompanying the user in straightening of the second sheet (14).

In another aspect, the invention is directed to a plurality of toothpaste capsules (10A), chained one to the other, the chaining including the first sheet (18) of each toothpaste capsules of the toothpaste capsules (10A) being disposed between each adjacent toothpaste capsules of the toothpaste capsules (10A),

thereby separation of the toothpaste capsule (10A) from the adjacent toothpaste capsule (10A) produces the opening (40).

In the figures and/or description herein, the following reference numerals (Reference Signs List) have been mentioned:
numeral 10A denotes the toothpaste capsule according to one embodiment of the invention;
10B: toothpaste capsule chain;
12: receptacle;
16: tail if sheet 14;
18: sheet, such as an aluminum foil, for covering;
20: tail of sheet 18;
22: toothpaste dose;
24: toothbrush;
40: opening;
42: package for rolled toothpaste capsule chain 10B;
50A,50B,50C,50D: internal walls of receptacle 12;
52A,52B,52C,52D: ends of internal walls 52A,52B,52C,52D respectively of receptacle 12;
54: end of sheet 14;
56: internal ceiling of receptacle 12;
58: cutting blade.

The foregoing description and illustrations of the embodiments of the invention have been presented for the purpose of illustration, and are not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

What is claimed is:

1. A toothpaste capsule, comprising:
a receptacle;
a first sheet, for covering said receptacle by sticking thereto, and being removable therefrom, for producing an opening to said receptacle; and
a toothpaste dose contained between said receptacle and said first sheet.

2. The toothpaste capsule according to claim 1, further comprising:
a second sheet, a first end thereof being connected to an end of one of internal walls of said receptacle, said second sheet for surrounding said toothpaste dose,
thereby removal of said first sheet allows straightening said second, to push said toothpaste dose out of said receptacle.

3. The toothpaste capsule according to claim 2, wherein said second sheet comprises a tail, for accompanying said straightening of said second sheet.

4. A plurality of toothpaste capsules according to claim 1, chained one to the other, the chaining comprising said first sheet of each toothpaste capsules of said toothpaste capsules being disposed between each adjacent toothpaste capsules of said toothpaste capsules, thereby separation of said toothpaste capsule from said adjacent toothpaste capsule produces said opening.
